(19) United States
(12) Patent Application Publication
(10) Pub. No.: US 2007/0020747 A1
(43) Pub. Date: Jan. 25, 2007

(54) Composition for eliminating ethidium bromide and use thereof

(76) Inventor: Fong-Ming Lu, Hualien City (TW)

Correspondence Address: HARNESS, DICKEY & PIERCE, P.L.C., P.O. BOX 8910, RESTON, VA, 20195, US

(21) Appl. No.: 11/184,950

(22) Filed: Jul. 20, 2005

(51) Int. Cl. C07H 21/04; C12N 1/08

(52) U.S. Cl. 435/270; 536/25.4

(57) ABSTRACT

The present invention is related to a composition and a method for eliminating ethidium bromide.

### FIELD OF THE INVENTION

The present invention is related to a composition and a method for eliminating ethidium bromide (EtBr).

### BACKGROUND OF THE INVENTION

The IUPAC nomenclature of EtBr is 3,8-diamino-5-ethyl-6-phenylphenthanridinium bromide. It is a flat molecule that illuminates reddish-brown fluorescence under ultraviolet. Because of its flat structure, EtBr can insert itself into the double helix structure of DNA or RNA (Patel and Canuel, 1976, Proc. Natl. Acad. Sci. USA., 73(10): 3343-3347).

EtBr is generally used as a dye in experiments of DNA or RNA. After electrophoresis, the gel containing DNA or RNA is embedded in EtBr solution to allow the insert of EtBr into DNA or RNA (Micklos and Freyer, 1990, DNA Science, Gold Spring Harbor Laboratory Press, Ch. 3, 49-50). The researcher can distinguish DNA or RNA with the reddish-brown color of EtBr under ultraviolet illumination. The binding of EtBr and DNA can affect or interfere the process of DNA replication, thereby causing DNA mutation. DNA mutation is one of the major factors of carcinogenesis, so that EtBr is well-known as a carcinogen or mutagen. Therefore, the special treatment is required to process the waste liquids or contaminants containing EtBR.

There are several available methods of processing waste liquids or disposals contaminated by EtBr, but each of them has disadvantages such as complicated process, use of strong acid or base, expensive, low effect of eliminating its toxicity and residue of the carcinogens. These methods and their disadvantages are discussed as follows:
(1) Dilution of the waste liquid: the waste liquid is diluted with water or alcohol and disposed in water sink directly. It is no effect to destroy EtBr and may contaminate the water source and the environment.
(2) Exposure to the sun: it is time consuming and requires personnel to guard the area or to erect warning signs.
(3) Collect contaminated disposals and waste liquid from labs and transport to specific institute: it is required such as storage space, trained personnel and a process room.
(4) Strong acid and strong base process (Micklos and Freyer, 1990, DNA Science, Gold Spring Harbor Laboratory Press, Prelab Notes, 256-257): these chemicals include strong acid and basic compositions such as KMnO₄, HCl, NaOH, sodium hypophosphorate and sodium nitrate. These methods not only use dangerous strong acid and basic compounds, but also require complicated and long processing (about 24 hours).
(5) Decontaminate waste liquid with commercial products: the products currently available on the marker can be divided into two types:
   a. Small package active carbon pellet or powder. These products employ collision effect to absorb EtBr and usually take more than 24 hours. These products also have the disadvantages of incomplete cleaning and small capacity.
   b. Positive ion-exchange resin. These products absorb EtBr on the resin by filtrating waste liquid through the core of the resin. These products have the disadvantages of high cost and complicated process (require pump motor).

### SUMMARY OF THE INVENTION

The present invention provides a composition for eliminating ethidium bromide comprises an oxidant.

The present invention also provides a method for eliminating EtBr comprises: (a) mixing an oxidants or a composition comprising oxidant with ethidium bromide; and (b) illuminating the mixture with light having wavelength less than 400 nm.

### BRIEF DESCRIPTION OF THE DRAWINGS

FIG. 1 shows the EtBr-eliminating activities of the oxidants detected by illuminating with UV. The use of oxidants such as lithium nitrate, sodium nitrite, the concentrations of oxidants used and UV illumination are indicated at the left of the figure. The 0 and 60 indicate the time period of illumination. The conditions of samples are also described in Table 1.

FIG. 2 shows an example of UV light apparatus used in the invention.

FIG. 3 shows the EtBr-eliminating activities of the oxidants detected by illuminating with UV. The conditions of samples are also described in Table 2.

### DETAILED DESCRIPTION OF THE INVENTION

It is surprisingly that using chemical oxidants along with light source of which wavelength no longer than 400 nm can facilitate the reaction of EtBr with said chemicals and destroy EtBr rapidly. The method of the invention is fast (less than 1 hour), simple, cost-effective, safe and can completely destory EtBr in large capacity. To perform the method of the invention, simply mix the oxidants of the invention with the target waste liquid at certain proportion, and then illuminate the mixture with light at wavelength shorter than 400 nm for a certain time. The processed waste liquid can be discarded in the water sink.

Accordingly, the invention provides a composition for eliminating EtBr comprises an oxidant. For example, but not limit, the oxidant is selected from the group consisting of sodium nitrite, aluminum nitrate, aluminum perchlorate, ammonium cerium nitrate, ammonium nitrate, ammonium persulfate barium peroxide, calcium nitrate, chromium nitrate, guanidine nitrate, iron nitrate, lithium nitrate magnesium nitrate, potassium iodate, potassium periodate, potassium persulfate, sodium bromate, sodium iodate, sodium nitrate, sodium persulfate, strontium nitrate, strontium peroxide, zinc nitrate and zinc peroxide. In a preferred embodiment, the oxidant is lithium nitrate or sodium nitrite.

The composition of this invention can destroy EtBr under the light having wavelength less than 400 nm. In an embodiment, the light is ultraviolet. In a preferred embodiment, the wavelength of the ultraviolet can be 180-380 nm, according to the concentration of the composition.

This invention also provides a method method for eliminating EtBr comprises: (a) mixing an oxidants or a composition comprising oxidant with ethidium bromide; and (b) illuminating the mixture with light having wavelength less than 400 nm. In a preferred embodiment, the light is ultraviolet. In particular, the composition is the composition described former

There is no limit for the oxidant used in the present invention, as long as it could destroy EtBr in solution when the mixture is illuminated with light of which wavelength is less than 400 nm. The oxidant comprises but is not limited to sodium nitrite, aluminum nitrate, aluminum perchlorate, ammonium cerium nitrate, ammonium nitrate, ammonium persulfate barium peroxide, calcium nitrate, chromium nitrate, guanidine nitrate, iron nitrate, lithium nitrate magnesium nitrate, potassium iodate, potassium periodate, potassium persulfate, sodium bromate, sodium iodate, sodium nitrate, sodium persulfate, strontium nitrate, strontium peroxide, zinc nitrate or zinc peroxide. In a preferred embodiment, the oxidant is lithium nitrate or sodium nitrite.

There is no limit for the volume of the oxidant used in the present invention, as long as it could destroy EtBr in solution when the mixture is illuminated with light of which wavelength is no longer than 400 nm. Using oxidants less than necessary require longer time period of illumination or shorter wavelength of light.

In a preferred embodiment, the oxidant in the mixture is at least 40-folds higher than the ethidium bromide in the molar concentration ratio. It means that the proportion of the oxidant and EtBr in the mixed solution is at least 40:1. In more preferred embodiment, the oxidant in the mixture is at least 90-folds higher than the ethidium bromide in the molar concentration ratio. It means that the proportion of the oxidant and EtBr in the mixed solution is 90:1.

There is no limit of the light source of the invention, as long as its wavelength is no longer than 400 nm. In a preferred embodiment, the light is ultraviolet. The duration of the illumination depends on the kind of the oxidant, the volume and concentration of the oxidant, the watts and wavelength of the light source, the volume of the waste liquid and the volume and concentration of EtBr in the waste liquid.

Persons skilled in the art can easily determine whether EtBr has been completely destroyed by using conventional detection methods such as ultraviolet examination, and thus can decide whether the duration of the illumination, the wavelength of the light, the watts of the light source or any other condition should be changed.

The following examples demonstrate the advantages of the invention. The examples below are non-limiting and are merely representative of various aspects and features of the present invention.

### EXAMPLES

### Example 1

### Using UV Examination to Evaluate the Efficacy of Eliminating EtBr

5M lithium nitrate (LiNO₃), 5M sodium nitrite (NaNO₂), and 5 g/ml EtBr solutions were prepared.

Group 1

7 glass bottles (about 7 ml of capacity) were prepared with solutions as described in Table 1. The bottle 1 was added 4 μl LiNO₃ and 996 μl EtBr, wherein the final concentration of LiNO₃ was 20 mM. The bottle 2 was added 100 μl LiNO₃ and 900 μl EtBr, wherein the final concentration of LiNO₃ was 500 mM. The bottle 3 was added 4 μl NaNO₂ and 996 μl EtBr, wherein the final concentration of NaNO₂ was 20 mM. The bottle 4 was added 100 μl NaNO₂ and 900 μl EtBr, wherein the final concentration of NaNO₂ was 500 mM. The bottle 5 was added 1 ml EtBr only. The bottle 6 was added 100 μl LiNO₃ and 900 μl EtBr. The bottle 7 was added 100 μl NaNO₂ and 900 μl EtBr.

The bottles 1-5 were illuminated with ultraviolet, which was 8 W and the wavelength was 180-280 nm. The samples were collected in the time point 0 and 60 minutes. The bottles 6 and 7 were not illuminated with ultraviolet and the samples were collected in the time point 60 minutes.

The condition of each sample was listed in Table 1.

TABLE 1
The conditions of samples in group 1.

| | Composition volume | | | Concentrations of | | | |
|---|---|---|---|---|---|---|---|
| No. of | (μl) | | | the oxidant before | | Collection | |
| the | 5M | 5M | 5 μ/ml | UV illumination | | time point | Label in |
| bottle | LiNO₃ | NaNO₂ | EtBr | (mM) | UV | (min) | |
| 1 | 4 | — | 996 | 20 | ◯ | 0, 60 | Li20 + UV |
| 2 | 100 | — | 900 | 500 | ◯ | 0, 60 | Li500 + UV |
| 3 | — | 4 | 996 | 20 | ◯ | 0, 60 | Na20 + UV |
| 4 | — | 100 | 900 | 500 | ◯ | 0, 60 | Na500 + UV |
| 5 | — | — | 1000 | 0 | ◯ | 0, 60 | UV |
| 6 | 100 | — | 900 | 500 | X | 60 | Li500 |
| 7 | — | 100 | 900 | 500 | X | 60 | Na500 |

Group 2

Except the wavelength of UV was changed to 280˜320 nm, all conditions were identical to group 1.

Group 3

Except the wavelength of UV was changed to 320˜380 nm, all conditions were identical to group 1.

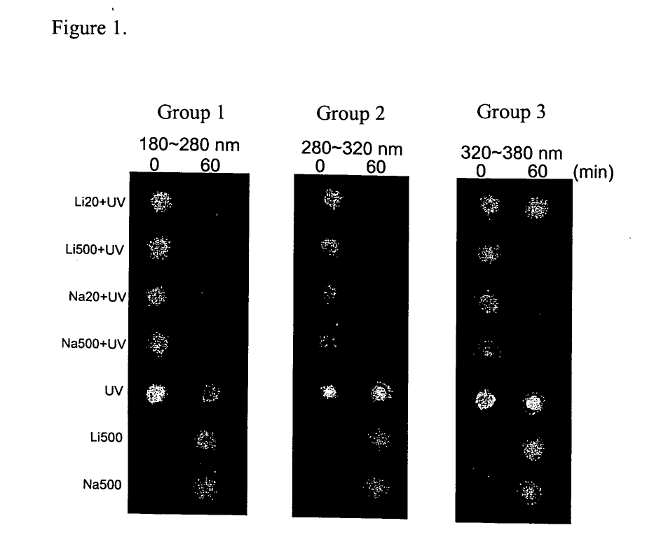

Each sample was collected 25 μl from the experimental bottle in the selected time point. There were 12 samples for each group, total 36 samples. These samples were dropped on the center of a filter paper (diameter 2.5 cm) and illuminated with UV to observe the elimination of EtBr. The result was shown in FIG. 1.

The result from these groups demonstrated EtBr in solutions added with lithium nitrate or sodium nitrite and illuminated with UV for 60 minutes was completely destroyed. It was shown that the fluorescence decayed or even disappeared. UV with wavelength 180˜280 nm showed the strongest effect (fluorescence completely disappeared); UV with wavelength 180˜280 nm showed modest effect whereas UV with wavelength 320˜380 nm was less effective. Samples that are only illuminated with UV but not added with lithium nitrate or sodium nitrite, or samples only added with lithium nitrate or sodium nitrite but not illuminated with UV, showed no or insignificant EtBr cleaning effect.

### Example 2

### Using UV Examination to Evaluate the Efficacy of Eliminating EtBr

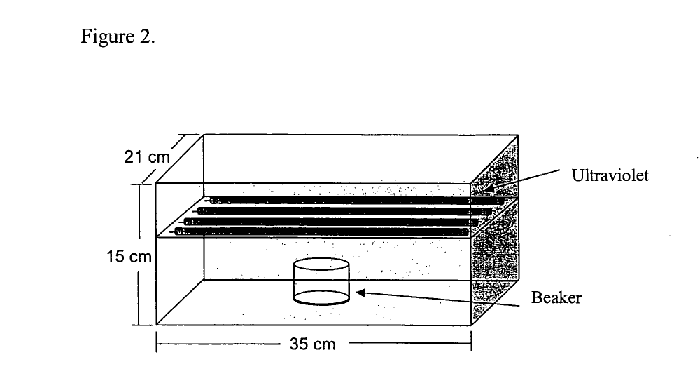

The UV light source was set up as shown in FIG. 2. The appearance of the light source was a rectangular lid and the size was 35 cm×21 cm×15 cm. Four parallel 8 W UV tubes were implanted on the top of the lid.

5 beakers were prepared. Beaker 1 and 2 were added 100 μl LiNO₃ and 9.9 ml EtBr, so that the final concentration of LiNO₃ was 50 mM. Beaker 3 and 4 were added 100 μl NaNO₂ and 9.9 ml EtBr, so that the final concentration of NaNO₂ was 50 mM. Beaker 5 was added 10 ml EtBr only. The beakers 1, 3 and 5 were illuminated with UV, and the samples were collected in the time points 0, 15, 30, 60 and 120 minutes. The beakers 2 and 4 were not illuminated with UV, and the samples were collected in the time point 120 minutes.

The condition of each sample was listed in Table 2.

TABLE 2
The conditions of samples.

| | | | | Concentrations of | | | |
|---|---|---|---|---|---|---|---|
| No. of | Composition (ml) | | | the oxidant before | | Collection | |
| the | 5M | 5M | 5 μ/ml | UV illumination | | time point | Label in |
| beaker | LiNO₃ | NaNO₂ | EtBr | (mM) | UV | (min) | |
| 1 | 0.1 | 0 | 9.9 | 50 | ◯ | 0, 15, 30, | Li + UV |
| | | | | | | 60, 120 | |
| 2 | 0.1 | 0 | 9.9 | 50 | X | 120 | Li |
| 3 | 0 | 0.1 | 9.9 | 50 | ◯ | 0, 15, 30, | Na + UV |
| | | | | | | 60, 120 | |
| 4 | 0 | 0.1 | 9.9 | 50 | X | 120 | Na |
| 5 | 0 | 0 | 10 | 0 | ◯ | 0, 15, 30, | UV |
| | | | | | | 60, 120 | |

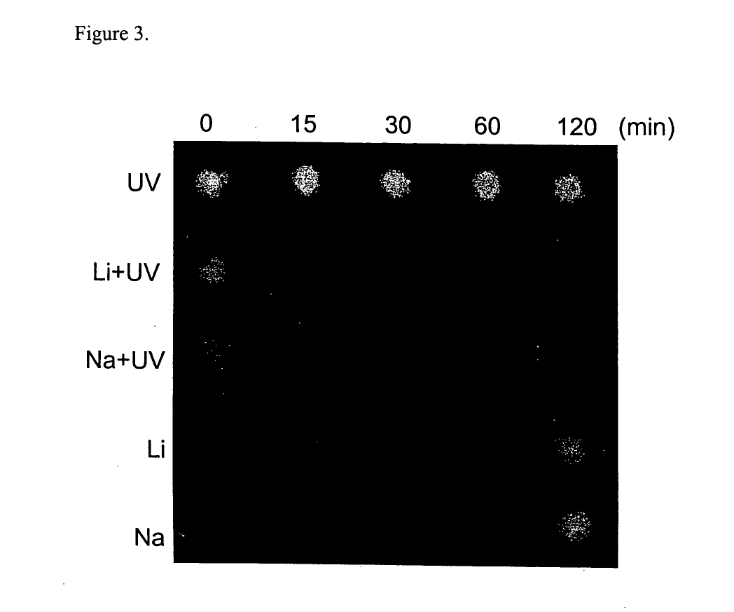

Each sample was collected 25 μl from the experimental beaker in the selected time point. There were 17 samples totally. These samples were dropped on the center of a filter paper (diameter 2.5 cm) and illuminated with UV to observe the elimination of EtBr. The result was shown in FIG. 3.

The result showed that EtBr were not destroyed (or insignificantly destroyed) in the samples illuminated with UV but not added with the oxidant LiNO₃ or NaNO₂ and the samples treated with oxidant LiNO₃ or NaNO₂ but not UV illumination. The fluorescence represented by EtBr in these samples could be clearly observed. To contrary, most EtBr in samples added with the oxidant LiNO₃ or NaNO₂ were destroyed after illuminating with UV for 15 minutes. After 60 minutes of UV illumination, almost all of EtBr in the samples were destroyed (no fluorescence could be detected).

The examples above clearly showed that combining the oxidant and ultraviolet of which wavelength less than 400 nm could facilitate the reaction between EtBr and the oxidant, so that EtBr could be rapidly destroyed.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The compositions and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A composition for eliminating ethidium bromide comprises an oxidant.

2. The composition of claim 1, wherein the oxidant is selected from the group consisting of sodium nitrite, aluminum nitrate, aluminum perchlorate, ammonium cerium nitrate, ammonium nitrate, ammonium persulfate barium peroxide, calcium nitrate, chromium nitrate, guanidine nitrate, iron nitrate, lithium nitrate magnesium nitrate, potassium iodate, potassium periodate, potassium persulfate, sodium bromate, sodium iodate, sodium nitrate, sodium persulfate, strontium nitrate, strontium peroxide, zinc nitrate and zinc peroxide.

3. The composition of claim 2, wherein the oxidant is lithium nitrate or sodium nitrite.

4. The composition of claim 1, which destroys ethidium bromide under the light having wavelength less than 400 nm.

5. The composition of claim 4, wherein the light is ultraviolet.

6. A method for eliminating ethidium bromide comprises:
(a) mixing an oxidants or a composition comprising oxidant with ethidium bromide; and
(b) illuminating the mixture with light having wavelength less than 400 nm

7. A method for eliminating ethidium bromide using the composition of claim 1.

8. The method of claim 6, wherein the oxidant is lithium nitrate or sodium nitrite.

9. The method of claim 6, wherein the oxidant in the mixture is at least 40-folds higher than the ethidium bromide in the molar concentration ratio.

10. The method of claim 9, wherein the oxidant in the mixture is at least 90-folds higher than the ethidium bromide in the molar concentration ratio.

11. The method of claim 6, wherein the light is ultraviolet.

12. The method of claim 7, wherein the light is ultraviolet.
